(19) United States
(12) Patent Application Publication
(10) Pub. No.: US 2012/0161796 A1
(43) Pub. Date: Jun. 28, 2012

(54) CO2 SENSING MATERIALS AND SENSORS INCORPORATING SAID MATERIALS

(76) Inventors: Peter Smith, Summertown (GB); Leon Cavanagh, Loughrea (IE)

(21) Appl. No.: 12/975,659

(22) Filed: Dec. 22, 2010

(51) Int. Cl. G01R 27/08; G01N 25/00; G01N 33/00

(52) U.S. Cl. 324/693; 252/408.1; 73/25.01

(57) ABSTRACT

A gas-sensitive material is disclosed for detecting CO₂ for use in semiconductor-based gas sensors. The gas-sensitive material may comprise one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof. The material may further comprise one or more oxides selected from the group CuO, Bi₂O₃, Sb₂O₃, Sb₂O₅, La₂O₃, Cr₂O₃, Fe₂O₃, NiO, and TiO₂, and any combination thereof. The material may further comprise one or more dopants such as Pt, Pd, Ag, Au or their compounds, and any combination thereof. In one embodiment, the material comprises a mixture of BaO-rich glass, BaWO₄, and WO₃. In another embodiment, the material comprises a mixture of at least one of BaCO₃ or BaO-rich glass in combination with at least one of WO₃ or W(OH)₆. Transducers and sensors incorporating such gas-sensitive materials are also disclosed.

### FIELD OF THE INVENTION

The disclosure herein relates to CO₂ sensing, for example using metal oxide semiconducting technology.

### BACKGROUND OF THE INVENTION

Accurate detection and measurement of gases is highly desirable for many reasons, including health and safety, environmental monitoring, and energy saving. However, it is not a straightforward task, particularly in the case of CO₂ measurement, which has often been performed by a non-dispersive infra-red (NDIR) technique. This optical method can be quite accurate and selective, but devices tend to be large, expensive, and to consume large operating power. This is because of the large housing and gas chamber or tubing, infra-red beam generation, mirrors, reflectors, optical filters and detectors, together with hand-assembly.

A further restriction is that optical devices are unsuited for use in hot, hazardous conditions such as are encountered in combustion environments. The current drive to make leaner engines and curtail harmful emissions has demanded the development of new exhaust gas sensors. At present, the technology underpinning such sensors is referred to as solid-state electrochemistry. This technology has delivered two oxygen sensors, the lambda and the broadband sensors, which are a main feature of automotive gas sensing techniques.

Electrochemical sensors can also detect CO₂, however the existing products provide an output which is related to the logarithm of the gas concentration and are thus better suited for detecting large shifts in CO₂ rather than the more modest changes required for indoor air quality applications. Slow response and recovery times, humidity interference, and high power consumptions have also been reported for these sensors.

Therefore much attention has been focused over the last few decades on the use of metal oxide-semiconducting (“MOS”) gas sensors. The basic principle of operation is the induction or transduction of a small change in electrical characteristics (conductivity, permittivity, or spectral impedance) of the material (either as a porous coating or a thin film), in response to the ingress/absorption/adsorption of the target gas. These MOS sensors have the inherent advantage of being small, long-life, low maintenance, inexpensive, are compatible with fully automated production techniques and can be integrated with semiconductor processing to offer added functionality. Greater integration also generally results in lower power, due to reduced parasitic capacitances, important for battery-operated applications. These oxide materials can be deposited on ceramic or plastic substrates to operate as stand-alone component sensors, where the conditioning electronics are provided externally in discrete form or by an ASIC. Alternatively, the sensor substrates may be assembled with an MCU or ASIC to form a sensor module (“two-chip” or module gas-sensor system). Alternatively the oxide materials may be deposited or formed on top of a silicon substrate, which may also contain some or all of the signal-processing circuitry to condition the output of the sensor (“single-chip” gas-sensor).

There have been some market successes in particular in automotive cabin air quality sensing where MOS sensors are deployed to sense pollution gases (CO, NOx) and in residential alarms for detecting CO and methane gas. However broader success of MOS and other polymer materials as gas sensors in the marketplace has been limited due to a variety of reasons—performance issues related to material stability, baseline drift, and cross-sensitivity of the sensor material to other non-target gases and humidity.

Although SnO₂ is the most widely deployed MOS material for gas-sensing, its problems with stability and humidity interference are well documented. Other efforts have focused on other gas-sensitive systems such as thick-film BaSnO₃, BaSnO3-Sb2O5, BaSnO₃—CuO, BaTiO₃, BaTiO₃—CuO. More recent work focuses on LaOCl, on thin-film BaTiO₃—CuO designs, on nanoparticulate homogeneous ternary oxides and on metal oxycarbonate systems. In the main, all show a positive increase in resistance in the presence of CO₂ but tend to conform to a logarithmic relationship with gas concentration, do not show the necessary discrimination in the dilute concentrations of 350-2000 ppm as required for indoor air quality applications and show a high humidity response. Indeed, the critical role played by humidity in the reaction mechanism responsible for CO₂ detection has been described. A recent introduction to the marketplace for CO₂ sensing is sensor product AB SB-AQ6A-00 from FIS Inc. (Japan). It is based on La-doped SnO2. The build is based on a suspended bead—this is less conducive to low-cost volume manufacture than is the more conventional thick-film planar build. The sum total of all of these efforts is inadequate performance to meet the market requirement.

Attempts have been cited in the literature on the deployment of MOS sensors in combustion atmospheres. In addition, the use of n-type homogenised BaSnO3 has been described and the use of SrTi₁₋ₓFeₓO_{3-δ} to detect oxygen changes has been described. Others have focussed on NOₓ detection describing the use of nanoparticulate BaₓWO_{y} or nanocrystalline doped-CeO₂ while others have focussed on p-type materials for sensing combined CO and oxygen. To date, commercial success in combustion environments has eluded MOS sensors.

This techniques described herein are directed towards providing an improved gas-sensitive material for sensing CO₂.

### SUMMARY OF THE INVENTION

As described herein, gas-sensitive materials from the systems, Ba—W—O, Ba—W—C—O or Ba—W—C—O—H, and transducers and sensors incorporating such materials are disclosed. These may offer significant advantages over the prior art, in terms of some or all of the following parameters:
(a) sensitivity in the dilute concentrations 300 ppm to 2000 ppm CO₂ typical of ambient environments, and/or
(b) 1-20 vol % CO₂ typical of combustion environments, and/or
(c) reduced humidity influence, and/or
(d) repeatability and reliability, and/or
(e) baseline stability over time.

Although not restricted to a particular mechanism underlying suitability of these materials, the advantageous gas sensing behaviour may be due to gas interaction on the n-p or n-n heterojunctions formed at the boundaries between the primary and the secondary phases.

In one embodiment, the techniques disclosed herein provide a gas-sensitive material for detecting CO₂ for use in semiconductor-based gas sensors, wherein the material comprises at least two different phases, at least one of which is a Ba-containing phase and at least one other of which is a W-containing phase. Examples of suitable Ba-containing phases include BaCO₃, BaO-rich glass, and BaWO₄. Examples of suitable W-containing phases include BaWO₄, WO₃, and W(OH)₆. Thus, a gas-sensitive material may comprise any one or more of BaCO₃, BaO-rich glass, and BaWO₄ phases in combination with any one or more of BaWO₄, WO₃, and W(OH)₆ phases, as long as the gas sensitive material comprises at least two different phases, i.e., as long as the gas-sensitive material comprises at least one Ba-containing phase and at least one W-containing phase that is a different phase from the Ba-containing phase.

It will also be understood that a gas-sensitive material may comprise more than one Ba-containing phase selected from BaCO₃, BaO-rich glass, and BaWO₄; and/or may comprise more than one W-containing phase selected from BaWO₄, WO₃, and W(OH)₆.

In one embodiment, a gas-sensitive material may be produced in any suitable manner by combining any one or more of BaCO₃, BaO-rich glass, BaWO₄, BaWO₄, WO₃, and W(OH)₆ and/or other materials including those optional materials disclosed herein under suitable conditions. A gas sensitive material may also be produced by using methods starting with one or more Ba-containing and/or W-containing precursor materials other than BaCO₃, BaO-rich glass, BaWO₄, BaWO₄, WO₃, and W(OH)₆. Examples of such other precursor materials for forming gas-sensitive materials include, but are not limited to, Ba-containing materials such as barium citrate and BaNO₃, and W-containing materials such as tungsten tetrachloride (WCl₄). In particular, such precursor materials may be exposed, alone or in combination with other materials as required, to conditions effective to result in a gas-sensitive material.

In one respect, disclosed herein is a gas-sensitive material for detecting CO₂ for use in semiconductor-based gas sensors, the gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

In one embodiment, the gas-sensitive material may further comprise one or more oxides, which may be present in the range of 0 to 30 wt %. The oxide/s may be in one embodiment selected from the group CuO, Bi₂O₃, Sb₂O₃, Sb₂O₅, La₂O₃, Cr₂O₃, Fe₂O₃, NiO, and TiO₂, and may include any combination thereof. Thus, a gas-sensitive material may further contain only one of these oxides, or may comprise any combination of two or more of these oxides.

In one embodiment, the gas-sensitive material may further comprise one or more dopants, preferably present at 0-10 wt %, and preferably selected from the group Pt, Pd, Ag, Au or their compounds, and may include any combination thereof. Thus, a gas-sensitive material may contain only one of these dopants, or may comprise any combination of two or more of these dopants.

In another embodiment, the gas-sensitive material may comprise a mixture of BaO-rich glass, BaWO₄, and WO₃.

In another embodiment, the gas-sensitive material may comprise a mixture of at least one of BaCO₃ or BaO-rich glass in combination with at least one of WO₃ or W(OH)₆.

In another embodiment, the gas-sensitive material may have grain sizes in the nano-particulate range of about 1 to 400 nm or in the micro particulate range of about 0.4 μm to 40 μm.

In another respect, the techniques disclosed herein provide a CO₂ gas-sensing transducer comprising a heating element and a sense element including a gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

In one embodiment, the gas-sensitive material may be on a ceramic substrate and is in the form of a co-planar array of interdigitated electrodes with the gas-sensitive material coated thereupon, and preferably the electrode spacing is in the range of 60 μm to 70 μm and the gas-sensitive material has a thickness in the range of 140 μm to 160 μm. Changes in the conductivity, resistance, capacitance or impedance of the sense element may be monitored to reflect changes in a gas concentration.

In one embodiment, the heating element comprises platinum.

In one embodiment, the gas-sensitive material is covered by a catalytically active oxide or precious metal material to provide increased stability and additional protection against nuisance gases.

In another respect, disclosed herein is a method for detecting changes in CO₂ concentration of an atmosphere with a transducer, the method comprising: providing a transducer comprising a heating element and a sense element including a gas-sensitive material that comprises one or more Ba-containing phases and one or more W-containing phases, at least one of the Ba-containing phases being different than at least one of the W-containing phases; and contacting the atmosphere with the gas-sensitive material of the sense element and detecting changes in CO₂ concentration of the atmosphere by measuring changes in at least one of the conductivity, resistance, capacitance or impedance of the sense element of the transducer. In this method, the one or more Ba-containing phases may comprise at least one of BaCO₃, BaO-rich glass, BaWO₄, or any combination thereof; and the one or more W-containing phases may comprise at least one of BaWO₄, WO₃, W(OH)₆, or any combination thereof. The method may be in one exemplary embodiment for detecting changes in CO₂ concentration in residential or automotive cabin air quality atmosphere, where the CO₂ concentration is in the range 0 to 3,000 ppm CO₂. The method may be in another exemplary embodiment for detecting changes in CO₂ concentration in a reducing atmosphere of an exhaust from a combustion engine in the range 1 to 20 vol % CO₂.

In another respect, disclosed herein is a method for producing a CO₂ gas-sensing transducer of any embodiment above, the method comprising providing a heating element and a plurality of electrodes for the transducer; and forming a sense element for the transducer by depositing gas-sensitive material upon the plurality of electrodes by a technique that comprises at least one of screen-printing, stencil printing, spin-coating, sputtering, ink-jet printing, or any combination thereof. In this method, the gas-sensitive material may comprise one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

In a further aspect, the invention provides a CO₂ sensor comprising: a transducer comprising a heating element and a sense element including a gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof. The CO₂ sensor may further comprise a drive interface adapted to provide a voltage across said sense element, a sense interface adapted to monitor an electrical parameter of the sense element, and a processor adapted to process the monitored parameter.

In one embodiment, the processor may be adapted to maintain the sense element at a temperature in the range of 350° to 650° C.

### BRIEF DESCRIPTION OF THE DRAWINGS

Other objects, advantages and novel features of the present invention will become apparent from the following detailed description of the invention, given by way of example only, when considered in conjunction with the accompanying drawings, in which

FIG. 1 is a high level block diagram of a gas sensing system of the invention;

FIG. 2 is a perspective view of a CO₂ gas sensor including a MOS sensor ceramic chip wire bonded to pins in a package base;

FIG. 3 is an electrical schematic of the sensor;

FIG. 4 is a plot of resistance vs. time for a CO₂ sensor described below in Example 1.

FIG. 5 is a plot showing response of the sensor of Example 1 to CO₂ at 2% and 5% exposure;

FIG. 6 is a plot showing response of a sensor described below in Example 2;

FIG. 7 is a diagram illustrating a bench assembly for testing in a combustion environment; and

FIG. 8 is a plot showing response of a sensor of Example 3 below to CO₂ in a combustion environment comprising N₂, 10% O₂, 3% H₂O at 650° C., with the CO₂ level varying from 0.25% to 4%.

### DETAILED DESCRIPTION OF THE EMBODIMENTS

FIG. 1 is a high level block diagram of a gas sensing system 1 having a sense element 2 adjacent a heater element 3. The system 1 may comprise a heater controller 4, a circuit 5 for gas sensor conditioning, and a microcontroller 6. The transducer consists of the two-terminal gas sensitive sense element 2 and the two-terminal heater element 3 which is controlled so as to maintain the sense element 2 at the optimum operating temperature. The sense element 2 may have an impedance that is modulated according to the concentration of the exposed gas. The gas sensor conditioning electronics 4-6 monitor variations in the sensor element 2 impedance. These resistance or impedance variations when combined with calibration algorithms give a measure of value of the target gas concentration. The heater controller 4 monitors the sense element 2 temperature and controls the heater 3 power so as to maintain optimum operating conditions. The micro controller 6 with non volatile memory (NVM) stores calibration coefficients determined at manufacturing and may implement a number of data correction algorithms.

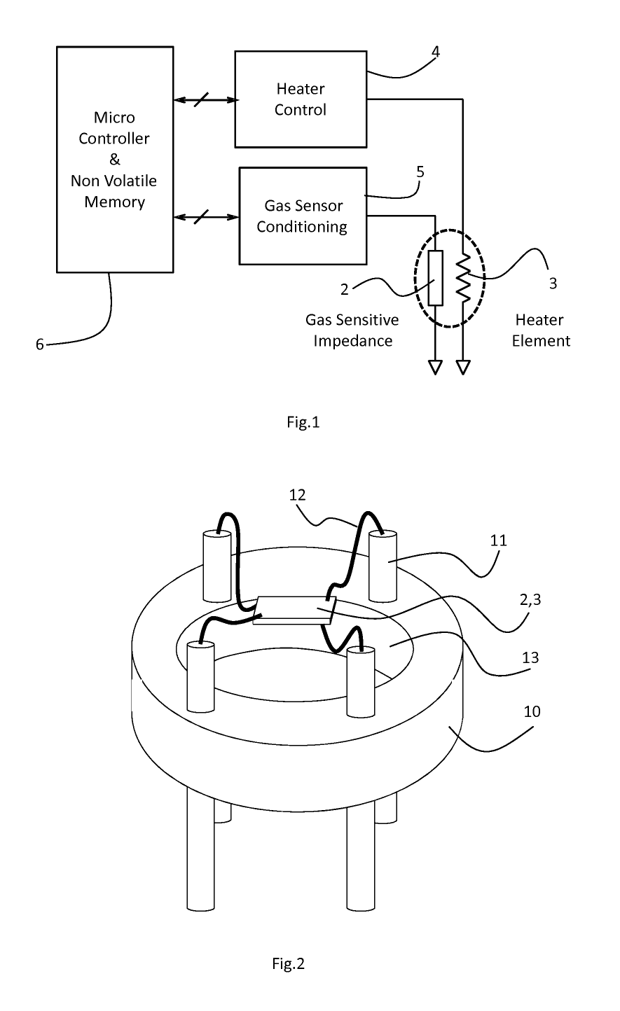

FIG. 2 shows one exemplary physical arrangement, in which a discrete transducer with the MOS sensor element 2 and the heater 3 is supported from a package base 10 having pins 11 linked to the transducer (sensor element 2 and heater 3) by wire bonds 12. The sense element 2 is on a heated sensor substrate which is thermally isolated from the package base 10 as it is suspended in midair. Heat loss is primarily by convection from the element 2 surface and by conduction through the bond wires 12. The electronic circuits 4-6 are on a separate PCB connected to the transducer via the pins 11. The base 10 is of plastics material and has a recess 13 under the transducer 2, 3. Other configurations of base are possible depending on the application. For example, the base may have a through hole aligned with the transducer for through-flow of a gas. It will be recognized that the techniques described herein are not limited to such physical arrangement and other base arrangements and circuitry may be used while still obtaining the benefits of the techniques disclosed herein.

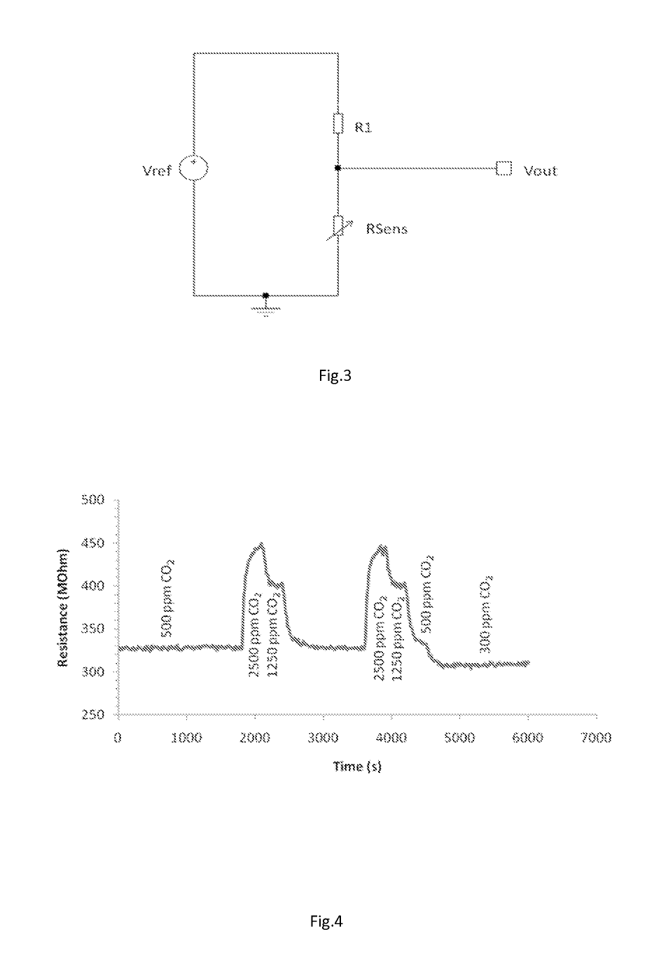

FIG. 3 shows an exemplary circuit diagram for a measurement circuit using a potential divider arrangement, in which the sense element 2 provides the resistance RSens. R1 is provided as the series resistor in this arrangement. It will be recognized that a wide range of circuit diagrams may be alternatively used while still obtaining the benefits of the techniques disclosed herein.

Interface Electronics 4, 5, 6 and Algorithms

The sense element 2 is thermally isolated, suspended in air by its bond-wires 12 (FIG. 2) and its temperature is controlled by means of the resistive heater element 3. The heater control circuit 4 directs current through the heater element 3. Feedback is obtained by monitoring the heater resistance and the embedded microcontroller 6 extrapolates the corresponding temperature using the known resistance-temperature profile of the heater element 3. In this way, the optimum operating conditions can be maintained. The gas sensor conditioning circuitry 5 monitors variations in the sense element 2 resistance and capacitance, and digitizes this data for further digital signal processing by the microcontroller 6.

Formulation and Demonstration of the CO₂ Gas-Sensitive Material:

The Metal-Oxide/Hydroxide/Carbonate gas-sensitive material is based on the Ba—W—O, the Ba—W—C—O or the Ba—W—C—O—H systems for the purposes of detecting CO₂. The gas-sensitive coating is comprised of at least one Ba-containing phase and a W-containing phase from the group (i) BaCO₃, BaO-rich glass, BaWO4, WO₃, W(OH)₆ or any combination of these, and 0-30 wt % of an oxide from the group (ii) CuO, Bi₂O₃, Sb₂O₃, Sb₂O₅, La₂O₃, Cr₂O₃, Fe₂O₃, NiO, TiO₂ to modulate the properties of conductivity and intrinsic drift, and 0-10 wt % of dopants from the group (iii) Pt, Pd, Ag, Au or their compounds to alter operating temperature, improve response kinetics and provide additional immunity to contaminant gases.

### Example 1

The gas-sensitive material was prepared by dry mixing commercial grade Barium Citrate powder (Aldrich, 325 mesh) with WO₃ powder (New Metals, Ceramic Grade) in the ratio 66 mol %:33 mol % on a roller mill The powdered mixture was then heated to 750° C. for 1 hour, which resulted in a gas-sensitive material including the following crystalline phases as determined by X-ray diffraction: BaWO₄ and WO₃. The material was then converted into a screen-printable ink by mixing it with a commercial ink vehicle, ESL 400, in a ball mill, such that the solids loading was 85 wt %. The gas sensor was then fabricated using a 250 μm thick 2 m×2 m aluminium oxide chip with on one side a serpentine platinum heater track and on the other side an interdgitated gold electrode pattern (65 μm electrode digit spacing), upon which a 150 μm thick layer was screen-printed using a 1202 DEK printer. The sensor chip was mounted onto a 4-pinned base by means of welding platinum wires between the chip bond pads and the pin heads (see FIG. 2).

Control of the sense element temperature was achieved by incorporating the heater into a constant-resistance Wheatstone bridge circuit arrangement. Using this set-up, the resulting sensor was heated to 650° C. for 1 hour and then the temperature reset to 600° C. The output of the sensor was measured in resistance mode. In the measurement circuit used, the sensor formed a resistive element in a potential divider circuit, in which the reference voltage (Vref) was a stable 1.5V and an appropriate series resistor (R1) chosen in order to drop a suitable voltage across the sensor (RSens). The output voltage (Vout) from the potential divider is amplified, digitised and logged by a microcontroller.

The sensor was subjected to pulses of 500 ppm, 2500 ppm and 1250 ppm CO₂ in a background of scrubbed air with a residual humidity level of 50% r.h. (see FIGS. 4 and 5) The following test sequence was used:
(i) 1200 seconds in static air
(ii) 600 seconds in flowing 500 ppm CO₂-balance air
(iii) 300 seconds in flowing 2500 ppm CO₂-balance air
(iv) 300 seconds in flowing 1250 ppm CO₂-balance air
(v) 1200 seconds in 500 ppm CO₂-balance air
(vi) 300 seconds in flowing 500 ppm CO₂-balance air
(vii) 300 seconds in flowing 2500 ppm CO₂-balance air
(viii) 300 seconds in flowing 500 ppm CO₂-balance air
(ix) 300 seconds in flowing 375 ppm CO₂-balance air
(x) 1200 seconds in static air.

FIGS. 4 and 5 show the response of the sensor described in Example 1 to CO₂. FIG. 4 is a plot showing the response of the sensor of Example 1 to various levels of CO₂ as encountered in indoor environments. The sensor is operating at 600° C. following conditioning at 650° C. The electrical resistance of the sensor is shown to increase as the CO₂ level increases. FIG. 5 is a plot showing the response of the sensor of Example 1 to high concentrations of CO₂. The sensor is operating at 600° C. following conditioning at 650° C. The electrical resistance of the sensor is shown to increase as the CO₂ level increases.

### Example 2

The gas-sensitive material was prepared by a wet chemistry route. A barium nitrate solution was prepared by dissolving 7.5 of BaNO₃ in 100 ml of water. To this was added, 300 ml of methylene chloride while continuously stirring. To the resulting emulsion, a solution of tungsten tetrachloride (7.31 g dissolved in 150 ml of 6 molar potassium hydroxide solution) was added with constant stiffing and a gel was formed. The gel was thoroughly washed with water until no more white silver chloride was formed by the addition of AgNO₃. The gel was dried at 50° C. overnight and subsequently fired at 800° C. for 4 hours. The calcined material was sieved through a 38 μm mesh and dispersed in an ESL 400 ink vehicle with a 80% solids loading to make a screen-printable ink. A sensor was fabricated along the same lines as in Example 1, and tested in CO₂ gas using the following test program:
(i) 60 seconds in static air
(ii) 300 seconds in flowing 500 ppm CO₂-balance air
(iii) 300 seconds in flowing 2500 ppm CO₂-balance air
(iv) 300 seconds in flowing 1250 ppm CO₂-balance air
(v) 600 seconds in 500 ppm CO₂-balance air
(vi) 700 seconds in static air

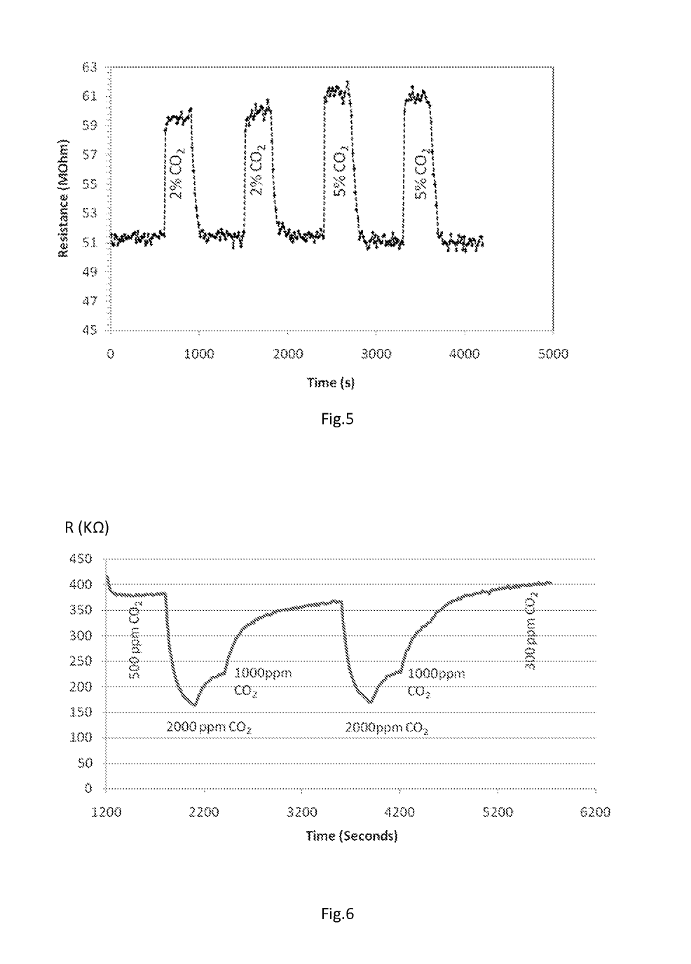

As in the test sequence of Example 1, the air supply used was scrubbed by passage through a filter system and all testing was carried out with a residual humidity level of 50% r.h. It should be noted that whereas the sensor in Example 1 exhibits an increase in resistance with increasing levels of CO₂, the sensor in Example 2 (as shown in FIG. 6) shows a decrease in resistance with increasing levels of CO₂. FIG. 6 is a plot showing the response of the sensor of Example 2 to various levels of CO₂ as encountered in indoor environments. The electrical resistance of the sensor is shown to decrease as the CO₂ level increases. The sensor was prepared as described in Example 2 and powered up to 600° C. without any prior conditioning at 650° C.

### Example 3

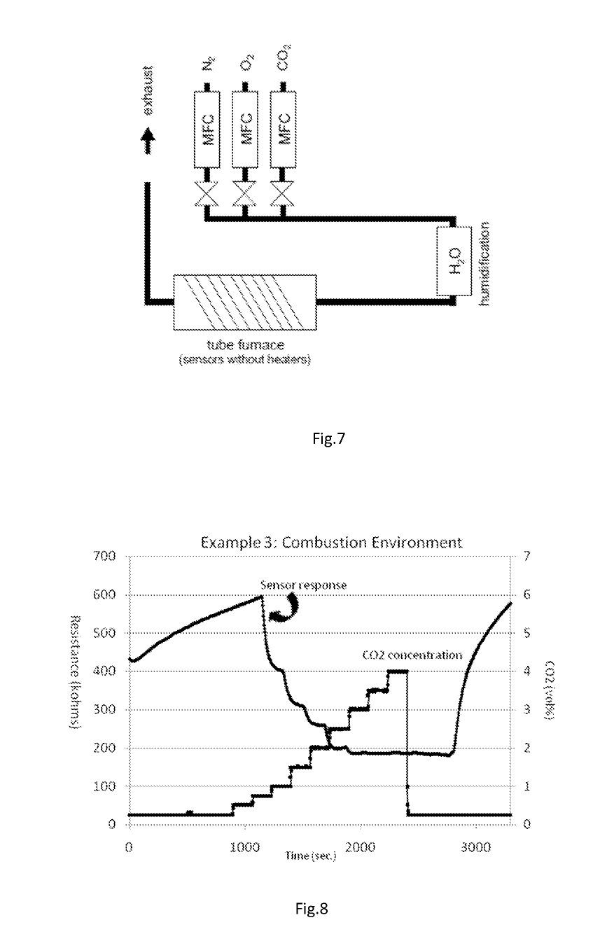

A sensor chip as prepared as in Example 1 but in this case, two meter long Pt wires were connected to the sensor electrodes and the chip was placed on an aluminium oxide tile which itself was positioned on a sectioned ceramic furnace tube, as shown in FIG. 7. This sectioned tube was then placed in an outer tube in a furnace which allowed controlled atmospheres. The mounted sensor chip was ‘passively’ heated by the furnace atmosphere to 650° C. in an atmosphere comprising a background composition of (in volume %) N₂, 10% O₂, 3% H₂O and 0.25% CO₂. As shown in FIG. 8, the CO₂ level was systematically increased in increments of 0.25% up to a maximum value of 4 vol % during the course of the test. Unlike in Example 1, the sensor was not conditioned by a firing to a higher temperature. It can be seen in FIG. 8 that the sensor responds to changes in CO₂ in the range 1 to 3 vol % with a decrease in resistance. Below 1 vol %, the sensor may be equilibrating with its surroundings or is ‘blind’ to dilute levels in the hot humid conditions of the combustion atmosphere. Above 3 vol %, it would appear that the sensor has ‘saturated’.

It will be appreciated that the techniques disclosed herein provide a sense material and sensor incorporating such material which as illustrated in the plots of FIGS. 4 to 6 and 8 exhibit a fast and reliable response to CO₂ concentration. Particular advantages are responsiveness—responds rapidly to sudden changes in CO₂ concentrations in the range 1-5% with good discrimination ability—and versatility—performs equally in benign residential environments and in hot humid combustion environments.

The invention is not limited to the embodiments described. For example, it is not limited to metal oxide semiconducting sensing technology but is applicable to field effect based gas sensing, mass balance gas sensing or surface acoustic wave gas sensing technologies.

What is claimed is:

1. A gas-sensitive material for detecting CO₂ for use in semiconductor-based gas sensors, the gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

2. The gas-sensitive material as claimed in claim 1, wherein the material further comprises at least one oxide.

3. The gas sensitive material as claimed in claim 1, wherein the material further comprises at least one oxide; and wherein said at least one oxide is present in the range of from about 0 to about 30 wt %.

4. The gas-sensitive material as claimed in claim 1, wherein the material further comprises one or more oxides; and wherein said one or more oxides are present in the range of from about 0 to about 30 wt %; and wherein said one or more oxides comprise at least one of CuO, Bi₂O₃, Sb₂O₃, Sb₂O₅, La₂O₃, Cr₂O₃, Fe₂O₃, NiO, TiO₂, or a combination thereof.

5. The gas-sensitive material as claimed in claim 1, wherein the material further comprises a dopant.

6. The gas-sensitive material as claimed in claim 1, wherein the material further comprises one or more dopants present in the range of from about 0 to about 10 wt %, the one or more dopants comprising at least one of Pt, Pd, Ag, Au, their compounds, or a combination thereof.

7. The gas-sensitive material as claimed in claim 1, wherein the material comprises a mixture of BaO-rich glass, BaWO₄, and WO₃.

8. The gas-sensitive material as claimed in claim 1, wherein the material comprises a mixture of at least one of BaCO₃ or BaO-rich glass in combination with at least one of WO₃ or W(OH)₆

9. The gas-sensitive material as claimed in claim 1, wherein the material has grain sizes in the nano-particulate range of from about 1 to about 400 nm or in the micro particulate range of from about 0.4 μm to about 40 μm.

10. A CO₂ gas-sensing transducer comprising a heating element and a sense element including a gas-sensitive material comprising one or more Ba-containing phases and one or more W-containing phases that are different than the one or more Ba-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

11. The transducer of claim 10, wherein the gas-sensitive material is on a ceramic substrate and is in the form of a co-planar array of interdigitated electrodes with the gas-sensitive material coated thereupon.

12. The transducer according to claim 10, wherein the gas-sensitive material is on a ceramic substrate and is in the form of a co-planar array of interdigitated electrodes with the gas-sensitive material coated thereupon; and wherein the electrode spacing is in the range of from about 60 μm to about 70 μm and the gas-sensitive material has a thickness in the range of from about 140 μm to about 160 μm.

13. The transducer according to claim 10, wherein the heating element comprises platinum.

14. The transducer according to claim 10, wherein the gas-sensitive material is covered by a catalytically active oxide or precious metal material effective to provide increased stability and additional protection against nuisance gases.

15. A method for detecting changes in CO₂ concentration of an atmosphere with a transducer, the method comprising:
providing a transducer comprising a heating element and a sense element including a gas-sensitive material that comprises one or more Ba-containing phases and one or more W-containing phases, at least one of the Ba-containing phases being different than at least one of the W-containing phases; and
contacting the atmosphere with the gas-sensitive material of the sense element and detecting changes in CO₂ concentration of the atmosphere by measuring changes in at least one of the conductivity, resistance, capacitance or impedance of the sense element of the transducer;
the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

16. The method of claim 15, the atmosphere being a residential or automotive cabin air quality atmosphere having a CO₂ concentration in the range of from about 0 to about 3,000 ppm CO₂.

17. The method of claim 15, the atmosphere being a reducing atmosphere of an exhaust from a combustion engine, the method comprising detecting changes in CO₂ concentration in the atmosphere in the range of from about 1 to about 20 vol % CO₂.

18. A method for producing a CO₂ gas-sensing transducer, comprising:
providing a heating element and a plurality of electrodes for the transducer; and
forming a sense element for the transducer by depositing gas-sensitive material upon the plurality of electrodes by a technique that comprises at least one of screen-printing, stencil printing, spin-coating, sputtering, ink-jet printing, or any combination thereof;
the gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

19. A CO₂ sensor comprising:
a transducer comprising a heating element and a sense element;
a drive interface adapted to provide a voltage across said sense element;
a sense interface adapted to monitor an electrical parameter of the sense element; and
a processor adapted to process the monitored parameter;
the sense element including a gas-sensitive material comprising one or more Ba-containing phases and one or more W-containing phases, at least one of the Ba-containing phases being different than at least one of the W-containing phases; the one or more Ba-containing phases comprising at least one of BaCO₃, BaO-rich glass, BaWO₄, or any combination thereof; and the one or more W-containing phases comprising at least one of BaWO₄, WO₃, W(OH)₆, or any combination thereof.

20. The sensor according to claim 19, wherein the processor is adapted to maintain the sense element at a temperature in the range of from about 350° to about 650° C.
